(19) United States
(12) Patent Application Publication
(10) Pub. No.: US 2009/0281436 A1
(43) Pub. Date: Nov. 12, 2009

(54) Low-frequency wireless heartbeat detector hanging over the ear

(76) Inventor: Chin-Yeh Hung, Taoyuan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 12/149,875

(22) Filed: May 9, 2008

(51) Int. Cl. A61B 5/02

(52) U.S. Cl. 600/504

(57) ABSTRACT

A low-frequency wireless heartbeat detector hanging over the ear includes: a) an ear-hanging unit hanging over the ear and having an internal portion and an external portion, the internal portion leaning against the internal side of the earlobe of a user, thereby creating a supporting effect; and b) a wireless heartbeat detector attached to the external portion of the ear-hanging unit, at least one part thereof leaning against the external side of the earlobe of the user such that a reflection type heartbeat-sensing element detects the slight blood change in the blood capillary of the earlobe created by heartbeat, thereby receiving the heartbeat impulse signal of the user which is then transmitted to a display unit having a receiver in a low-frequency wireless transmission way.

### BACKGROUND OF THE INVENTION

1. Field of the Invention

The invention relates to a heart rate monitoring device and particularly to a heartbeat detector whose sensing unit hangs over the ear and detects the signal of the flow change of blood within blood capillary. Meanwhile, the signals detected are transmitted to a display unit in a low frequency so as to achieve a convenient operation and an accurate heartbeat measurement.

2. Description of the Related Art

Conventionally, people's heart rate remarkably varies before and after people take exercise. During exercise, measuring the variables is very important. To take exercise properly and advantageously, the heart rate must increase to 60% of the maximum rate; however, if the heart rate increases to the maximum rate during exercise, it will do harm to the exerciser. Thus, a user-friendly and precise heart rate measurement device is very important for taking exercise to keep fit.

Next, FIG. 1 shows an electrode belt of heart rate monitor disclosed in U.S. Pat. No. 6,553,247, in which a heart rate monitoring unit 60 is bound on the chest with a belt 61 to monitor the heart rate on and on and wirelessly transmit a signal to a receiver 70 bound around the wrist. However, it is found that the patent is poor with the following conditions when being applied.

1. Generally, the heart rate monitoring unit 60 is tightly bound with the belt 61 on the chest to keep close to; however, the lung capacity increases during exercise and thus the belt bound up on the chest causes a user to be uncomfortable and breathe hard.

2. Female chest contour is bigger, so the heart rate monitoring unit 60 cannot stay fully close to the chest, thereby the accuracy of monitoring decreasing.

3. To accurately measure the heart rate, the heart rate monitoring unit 70 cannot be bound outside the clothes but inside the clothes and close to the chest, so the belt 71 is placed certainly after the clothes is taken off, which might be somewhat inconvenient to males but may be implemented none the less; however, it is very inconvenient to females to bind up the belt 71 in a public place; thus, the conventional belt is not properly used by the female.

In addition, there is a conventional heartbeat sensor disposed on a wristwatch. The user may place his fingertip or his thumb on the heartbeat sensor for measuring his heartbeat. However, this conventional unit is not suitable for use during the exercise sessions since the large movement will change the detected signals. Meanwhile, the finger has to stay on the sensor in a compressed way for a problemfree measurement. This is very impractical.

Moreover, another conventional heartbeat measurement way is illustrated in FIG. 2. According to the design of FIG. 2, a pair of sensing electrodes 71, 72 is positioned at both internal sides of an earlobe clip 70. The earlobe clip 70 is connected with a connecting cable 80. The detected voltage signals are transmitted to a host computer (not shown) via a connector 81, thereby displaying the heartbeat value or signals. However, this detection will be hindered by the connecting cable 80 when the user takes exercise. Moreover, it is not suitable for an outdoor use and it is not easy to take outdoors. Therefore, this requires further improvement.

Consequently, how to develop a heartbeat detector with low power consumption, a miniaturized structure, a convenient use and an accurate measurement is the main topic of the invention.

### SUMMARY OF THE INVENTION

A primary object of the invention to provide a low-frequency wireless heartbeat detector hanging over the ear which conducts the transmission in a low-frequency power and is miniaturized such that it can hang over the ear, thereby achieving a light product, a convenient carrying without great burden. Meanwhile, it is suitable for male and female use. Moreover, it won't affect the mobility and safety of the user.

Another object of the invention to provide a low-frequency wireless heartbeat detector hanging over the ear that closely leans against the earlobe without sliding movement, thereby achieving an accurate measurement of the tiny infrared reflection change of the blood flow within the blood capillary caused by each heartbeat.

A further object of the invention to provide a low-frequency wireless heartbeat detector hanging over the ear that begins with an automatic detection of the heartbeat value when it hangs over the ear and stops an automatic detection when removed, thereby saving the electric power, avoiding unnecessary waste and prolonging the service life of the product.

In order to achieve the above-mentioned objects, a low-frequency wireless heartbeat detector hanging over the ear in accordance with the invention includes:

a) an ear-hanging unit hanging over the ear and having an internal portion and an external portion, the internal portion leaning against the internal side of the earlobe of a user, thereby creating a supporting effect; and

b) a wireless heartbeat detector attached to the external portion of the ear-hanging unit, at least one part thereof leaning against the external side of the earlobe of the user such that a reflection type heartbeat-sensing element detects the slight blood change in the blood capillary of the earlobe created by heartbeat, thereby receiving the heartbeat impulse signal of the user which is then transmitted to a display unit having a receiver in a low-frequency wireless transmission way.

### BRIEF DESCRIPTION OF THE FIGURES

The accomplishment of this and other objects of the invention will become apparent from the following descriptions and its accompanying figures of which:

FIG. 1 is a schematic drawing illustrating the structure of a device disclosed in U.S. Pat. No. 6,553,247;

FIG. 2 is a perspective view of a conventional heartbeat detection clip;

FIG. 3A is a perspective view of a first embodiment of the invention that is viewed from outside to inside in an oblique manner;

FIG. 3B is a perspective view of the first embodiment of the invention that is viewed from inside to outside in an oblique manner;

FIG. 4A is a perspective view of a second embodiment of the invention that is viewed from outside to inside in an oblique manner;

FIG. 4B is a perspective view of the second embodiment of the invention wherein the wireless heartbeat detector is swiveled outwards;

FIG. 5 is a perspective view of the invention hanging over the ear of a user;

FIG. 6 is a schematic drawing in direction of 5-5 in FIG. 5;

FIG. 7 is a block diagram of a circuit according to the invention;

FIG. 8 is an application view of the invention; and

FIG. 9 is another application view of the invention.

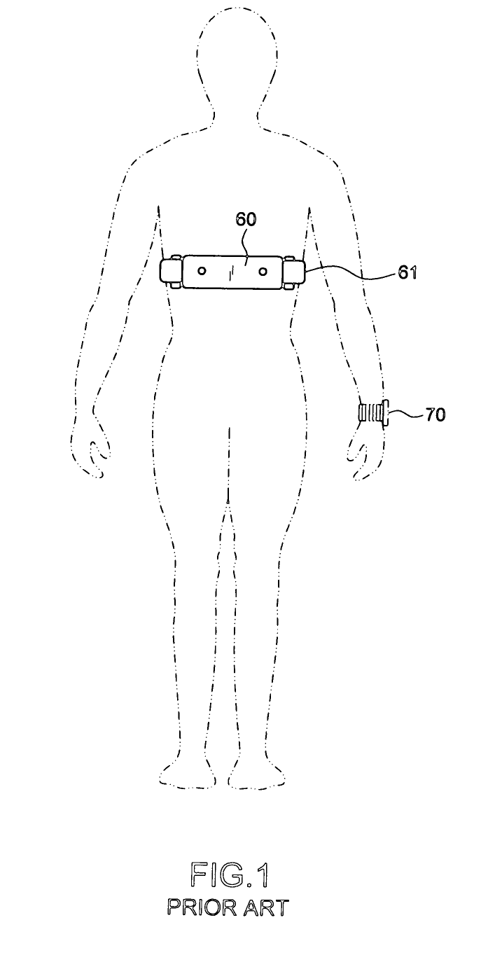

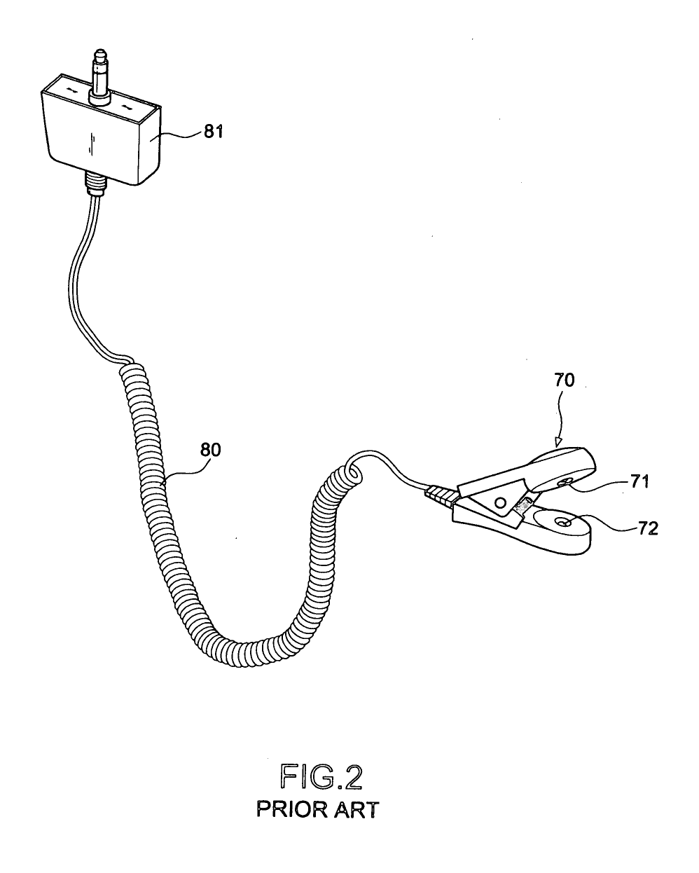

### DETAILED DESCRIPTION OF THE PREFERRED EMBODIMENT

Now, the present invention will be described more specifically with reference to the following embodiments. It is to be noted that the following descriptions of preferred embodiments of the invention are presented herein for purpose of illustration and description only; it is not intended to be exhaustive or to be limited to the precise form disclosed.

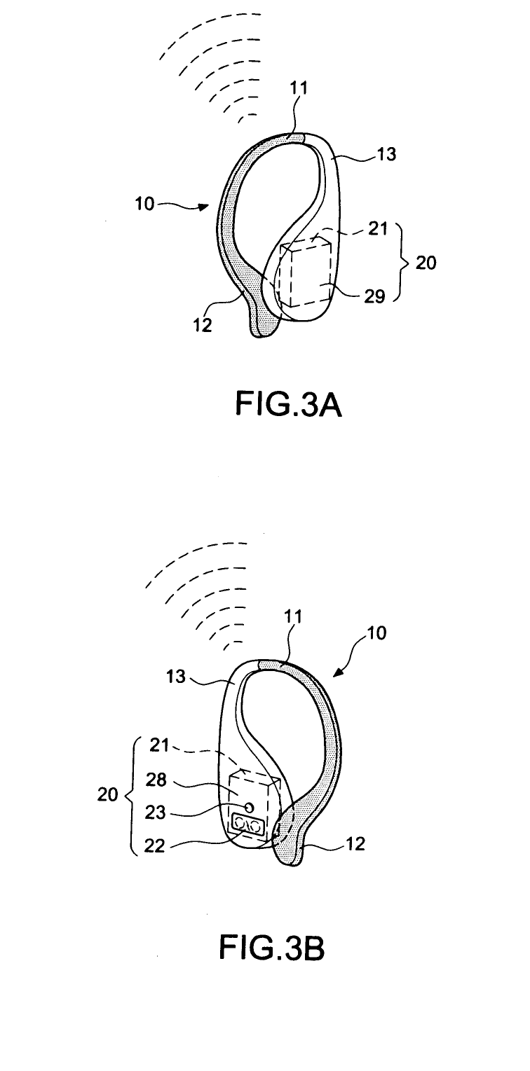

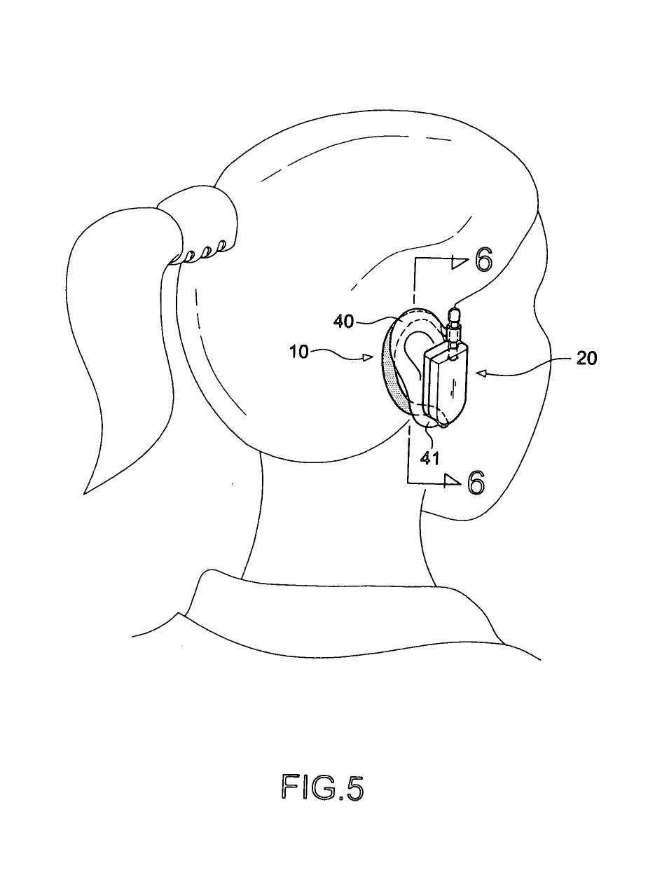

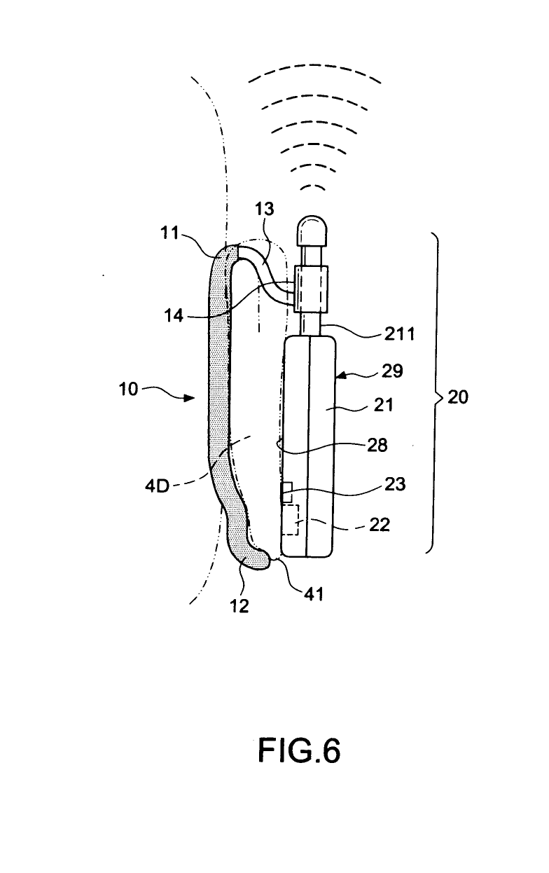

First, with reference to FIGS. 3A, 3B and 5 through 7, a first embodiment of the invention includes an ear-hanging unit 10. The ear-hanging unit 10 consists of a hanging portion 11 hanging over the ear 40 of a user, an internal portion 12 extending from the hanging portion 11 downwards with its bottom end approaching to the internal side of the earlobe 41, and an external portion 13 extending from the hanging portion 11 outwards to the external side of the ear 40. The above-mentioned ear-hanging unit 10 is formed in shape of the hanging part of an earphone. According to a preferred embodiment, the internal portion 12 of the ear-hanging unit 10 leans against the internal side of the earlobe 41 so as to achieve a supporting effect. In other words, the internal portion 12 and a wireless heartbeat detector 20 create an internal part and an external part to fulfill the clamping function. This function will be described hereinafter.

The above-mentioned wireless heartbeat detector 20 is attached to the external portion 13 of the ear-hanging unit 10. Moreover, at least one part thereof leans against the external side of the earlobe 41 of the user. Therefore, it is different from the earphone in shape. The earphone includes an earplug inserted into the opening of the outer ear. However, the wireless heartbeat detector 20 in accordance with the invention includes at least one portion leaning against the external side of the earlobe 41. Likewise, the internal portion 12 of the ear-hanging unit 10 leans against the internal side of the earlobe 41. In this way, an internal part and an external part are created to fulfill the clamping function.

According to the embodiment illustrated in FIGS. 3A and 3B, the wireless heartbeat detector 20 and the ear-hanging unit 10 are integrally formed. In other words, the housing 21 of the wireless heartbeat detector 20 can directly fit into the ear-hanging unit 10 with elements at the internal side 28 exposing to the outside.

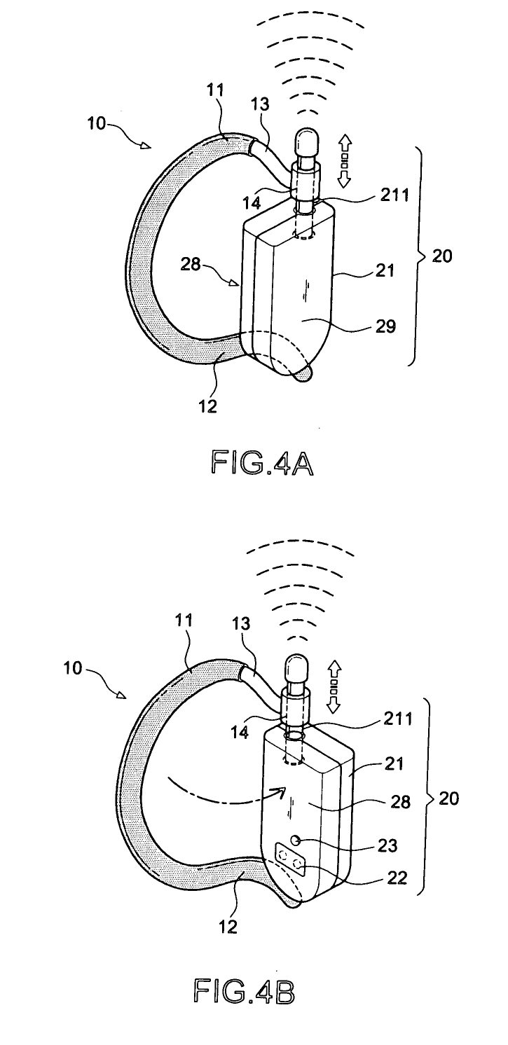

FIGS. 4A and 4B are perspective views of a second embodiment of the invention. The structure that is identical to that of the first embodiment is marked with the same reference signs. The difference lies in that the wireless heartbeat detector 20 and the ear-hanging unit 10 are separately formed and then attached to each other. According to the preferred embodiment, the external portion 13 of the ear-hanging unit 10 is provided with a cylindrical member 14. The wireless heartbeat detector 20 includes a stem 211 disposed at the top end of the housing 21 and liftable within the cylindrical member 14. In this way, the height of the housing 21 is adjustable according to different position of the earlobe 41 of the users. Furthermore, the housing 21 is pivotally attached to the stem 211. As shown in FIG. 4B, the housing 21 may rotated about the stem 211 at a certain angle. In this way, when the ear-hanging unit 10 hangs over the ear 40 of the user, the housing 21 may be inverted to lean against the earlobe 41 (see FIG. 4A). Thus, a convenient and comfortable use is achieved.

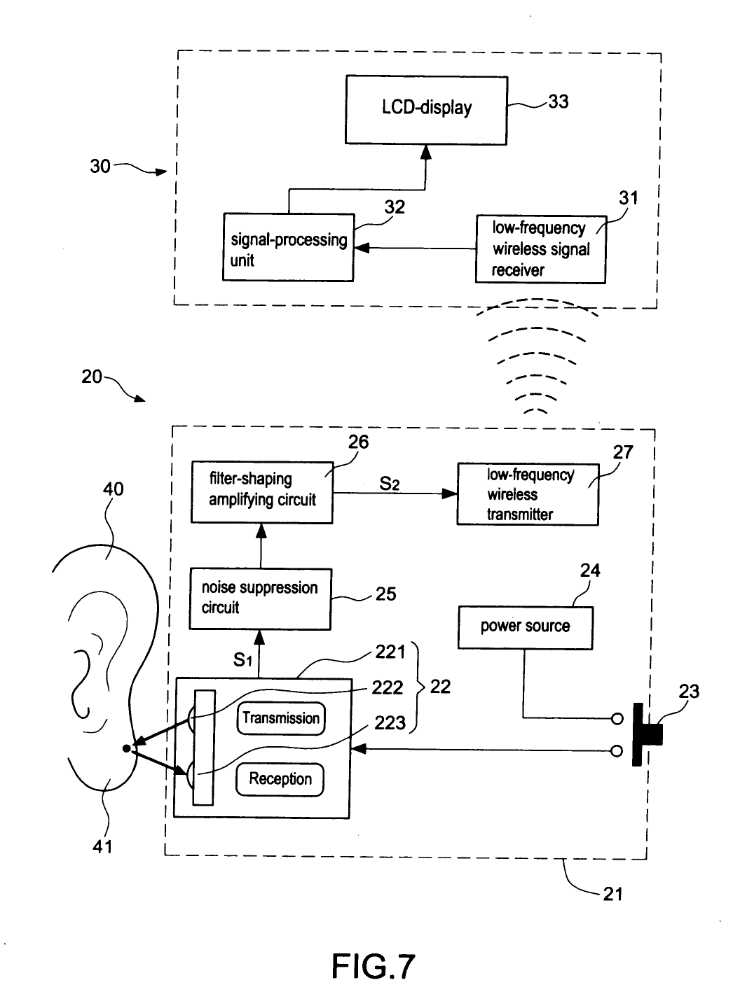

No matter if the above-mentioned two embodiments are integrally formed or assembled together, the configuration and the features of the wireless heartbeat detector 20 of both embodiments of the invention are the same. A circuit diagram of a wireless heartbeat detector 20 in accordance with the invention is shown in FIG. 7. The wireless heartbeat detector 20 includes a housing 21, a reflection type heartbeat-sensing element 22, a power source 24, a sensitive switch 23, a noise suppression circuit 25, a filter-shaping amplifying circuit 26 and a low-frequency wireless transmitter 27.

The housing 21 formed in match of the shape of the ear 40 includes an internal side 28 leaning against the ear 40 and an external side opposing to the internal side 28.

The reflection type heartbeat-sensing element 22 disposed within the housing 21 includes an infrared transmitting terminal 222 on a control circuit board 221 and a receiving terminal 223 in alignment with the infrared transmitting terminal 222. The infrared transmitting terminal 222 and the receiving terminal 223 are exposed on the internal side 28 of the housing 21 and lean against the external side of the earlobe 41 for detecting the slight blood change in the blood capillary of the earlobe 41 created by heartbeat. In this way, the heartbeat impulse signal of the user may be received. According to the embodiment, the infrared transmitting terminal 222 is an infrared light-emitting diode while the receiving terminal 223 is a receiving transistor. The heartbeat impulse signals can be measured when the infrared ray passes through the blood vessel of the skin and the reflection signals are received by the receiving transistor.

The power source 24 is positioned within the housing 21 for supplying the power required by the wireless heartbeat detector 20. According to this embodiment, the power source 24 is a mini-battery.

The sensitive switch 23 is positioned at the internal side 28 of the housing 21 for controlling the on/off state of the reflection type heartbeat-sensing element 22. According to this embodiment, the sensitive switch 23 is disposed at the side of the reflection type heartbeat-sensing element 22 (see FIGS. 3B and 4B). However, its installation position should not be restricted thereto. In other words, the sensitive switch 23 can be disposed at the center of the reflection type heartbeat-sensing element 22 as well, that is, at the center part between the infrared transmitting terminal 222 and the receiving terminal 223. The reason lies in that the detection of the heartbeat begins when the internal side 28 of the housing 21 is in contact with the earlobe 41. An automatic shutdown takes place when the housing 21 is removed from the earlobe 41. In this way, the consumption of the power source 24 can be reduced for prolonging the service life of the product.

The noise suppression circuit 25 and the filter-shaping amplifying circuit 26 are disposed within the housing 21 and electrically coupled to the reflection type heartbeat-sensing element 22. The human heartbeat signal detected by the reflection type heartbeat-sensing element 22 in the principle of the reflection is an impulse signal S1 that is a weak analog signal required to be converted into -a digital signal S2 by means of the noise suppression circuit 25 and the filter-shaping amplifying circuit 26 for the use of a signal-processing unit 32.

The low-frequency wireless transmitter 27 is disposed within the housing 21 for transmitting the heartbeat, signal of the filter-shaping amplifying circuit 26 to a display unit 30 having a low-frequency wireless signal receiver 31 for displaying the heartbeat signal.

One of the features of the invention lies in that the low-frequency wireless transmitter 27 and the low-frequency wireless signal receiver 31 create a radio frequency module for transmitting signals that is different from the conventional wireless earphone employing the high-frequency transmission or the blue-tooth device. The conventional apparatus conducts the transmission under a high frequency of 2.4 GHz with a power of 200 megawatt. This does not meet the requirement of low frequency. Moreover, it is feared that the electromagnetic high-frequency waves do harm to people. In addition, the cost of the blue-tooth chips is very high. Besides, the power source for the wireless products is the built-in small battery. Therefore, the amount of the consumption power of the electronic module of the wireless products will affect the service time of the product. Accordingly, the low-frequency wireless transmitter 27 of the invention conducts the transmission of the heartbeat signals in the frequency range between 10 MHz and 80 MHz unlike the conventional blue tooth apparatus with high-frequency power. Furthermore, the volume of the single chip is reduced. Meanwhile, the low-frequency transmission prevents the high-frequency power from potentially doing harm to the human body. Moreover, the radio frequency module of the invention is formed in small dimensions and operated in the low-voltage and low-frequency mode so that an easy application to different products is achieved.

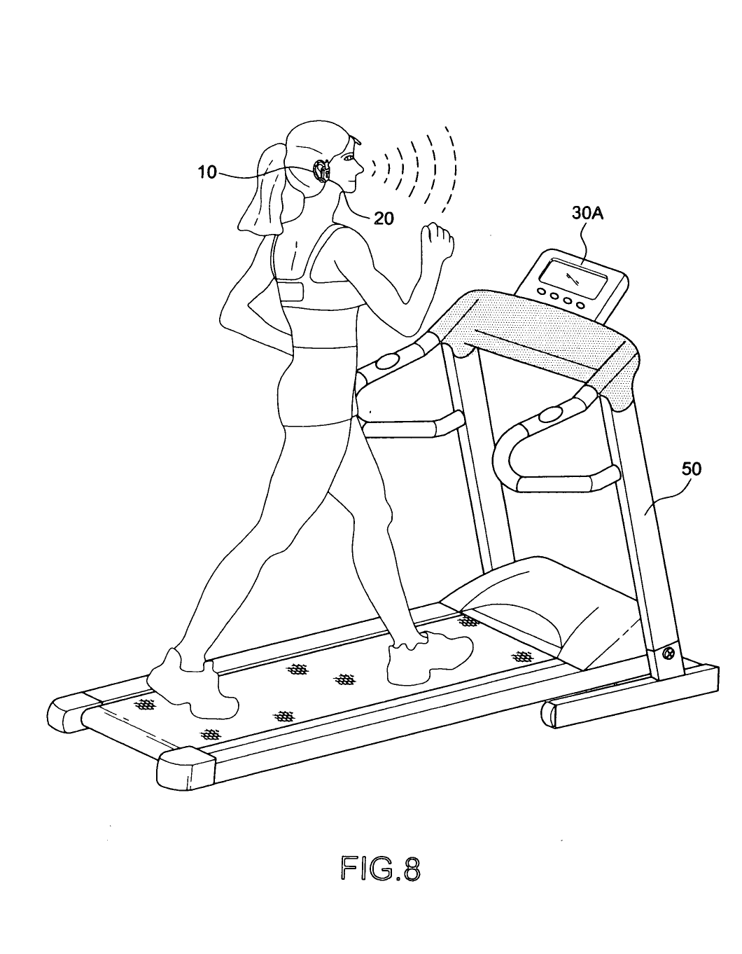

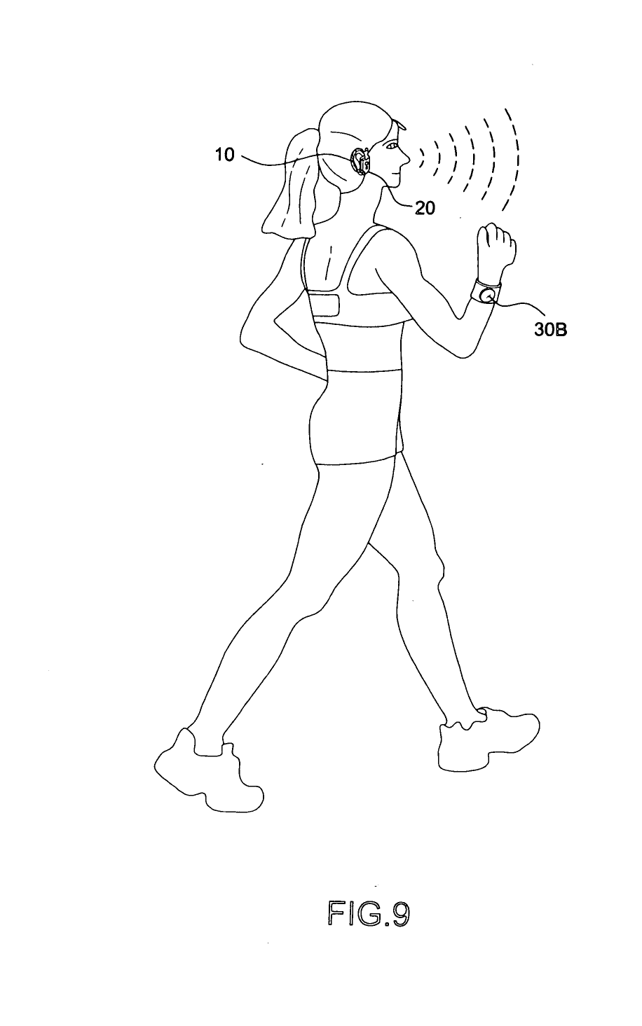

In order to reduce the volume and the weight of the wireless heartbeat detector 20 for a practical carrying and use and to ensure a continuous detection of the heartbeat state of the user during the exercise session, the housing 21 is formed in shape of the earphone. Meanwhile, the reflection type heartbeat-sensing element 22 at the internal side 28 of the housing 21 leans closely against the earlobe 41 for detecting the heartbeat signal of the user. Moreover, the display unit occupying more space is removed and replaced by a peripheral electronic product. According to the radio frequency module of the invention, the measured heartbeat signals are transmitted by a low-frequency wireless transmitter 27 to a display unit 30 having a low-frequency wireless signal receiver 31. With the progress of the technology, many electronic products are provided with a display panel such as a control panel 30A of an exercise apparatus 50 illustrated in FIG. 8, an exercise type wrist watch 30B as illustrated in FIG. 9, a cellular phone, an MP3/4-player, etc. These belong to the prior art so that no further descriptions thereto are given hereinafter. As a result, a wireless transmission can be achieved only when these electronic products are provided with the low-frequency wireless signal receiver 31.

In addition to the LCD-display 33, the display unit 30 further includes a signal-processing unit 32 (see FIG. 7) with prearranged program or circuit for operating and processing the signals inputted by the low-frequency wireless signal receiver 31 that are then transmitted to the LCD-display 33 for the user to read and use.

As shown in FIG. 8, the wireless heartbeat detector 20 may hang over the ear of a treadmill's user and transmit the detected heartbeat signals to the control panel 30A without annoying the treadmill's user.

As shown in FIG. 9, the wireless heartbeat detector 20 may transmit the detected heartbeat signals to the exercise type wrist watch 30B when the user takes exercise outdoors. Thus, a convenient and ergonomic use is achieved without affecting the exercise.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A low-frequency wireless heartbeat detector hanging over the ear, comprising:
a) an ear-hanging unit having a hanging portion hanging over the ear of a user, an internal portion extending from the hanging portion downwards with its bottom end approaching to the internal side of the earlobe, and an external portion extending from the hanging portion outwards to the external side of the ear; and
b) a wireless heartbeat detector attached to the external portion of the ear-hanging unit, at least one part thereof leaning against the external side of the earlobe of the user, the wireless heartbeat detector having:
i) a housing formed in match of the shape of the ear and having an internal side leaning against the ear and an external side opposing to the internal side;
ii) a reflection type heartbeat-sensing element disposed within the housing and having an infrared transmitting terminal on a control circuit board and a receiving terminal in alignment with the infrared transmitting terminal, the infrared transmitting terminal and the receiving terminal being exposed on the internal side of the housing and leaning against the external side of the earlobe for detecting the slight blood change in the blood capillary of the earlobe created by heartbeat, thereby receiving the heartbeat impulse signal of the user;
iii) a power source positioned within the housing for supplying the power required by the wireless heartbeat detector;
iv) a sensitive switch positioned at the internal side of the housing for controlling the on/off state of the reflection type heartbeat-sensing element;
v) a noise suppression circuit and the filter-shaping amplifying circuit disposed within the housing and electrically coupled to the reflection type heartbeat-sensing element; and
vi) a low-frequency wireless transmitter disposed within the housing for transmitting the heartbeat signal of the filter-shaping amplifying circuit to a display unit having a low-frequency wireless signal receiver for displaying the heartbeat signal.

2. The low-frequency wireless heartbeat detector hanging over the ear as recited in claim 1, wherein the low-frequency wireless transmitter and receiver conduct the transmission of the heartbeat signals in the frequency range between 10 MHz and 80 MHz.

3. The low-frequency wireless heartbeat detector hanging over the ear as recited in claim 1, wherein the wireless heartbeat detector and the ear-hanging unit are integrally formed.

4. The low-frequency wireless heartbeat detector hanging over the ear as recited in claim 1, wherein the wireless heartbeat detector and the ear-hanging unit are separately formed and then attached to each other.

5. The low-frequency wireless heartbeat detector hanging over the ear as recited in claim 4, wherein the external portion of the ear-hanging unit is provided with a cylindrical member, and the wireless heartbeat detector includes a stem disposed at the top end of the housing and liftable within the cylindrical member.

6. The low-frequency wireless heartbeat detector hanging over the ear as recited in claim 5, wherein the housing and the stem are pivotally attached to each other.

7. The low-frequency wireless heartbeat detector hanging over the ear as recited in claim 1, wherein the display unit includes a control panel of an exercise apparatus with a low-frequency wireless signal receiver and a LCD-display.

8. The low-frequency wireless heartbeat detector hanging over the ear as recited in claim 1, wherein the display unit includes an exercise type wrist watch with a low-frequency wireless signal receiver and a LCD-display.
